(19) United States
(12) Patent Application Publication
(10) Pub. No.: US 2026/0363670 A1
(43) Pub. Date: Nov. 19, 2026

(54) DETECTING LUNG DYSFUNCTION USING AUTOMATED ULTRASOUND MONITORING

(71) Applicant: UNIVERSITY OF WASHINGTON, Seattle, WA (US)

(72) Inventors: Adeyinka Adedipe, Seattle, WA (US); Michael R. Bailey, Seattle, WA (US); Bryan Cunitz, Seattle, WA (US); Ross Kessler, Seattle, WA (US); Tatiana D. Khokhlova, Seattle, WA (US); Daniel F. Leotta, Seattle, WA (US); Adam D. Maxwell, Seattle, WA (US); Jeff Thiel, Seattle, WA (US); Gilles Thomas, Seattle, WA (US); Jane Hall, Seattle, WA (US)

(73) Assignee: UNIVERSITY OF WASHINGTON, Seattle, WA (US)

(21) Appl. No.: 19/130,067

(22) PCT Filed: Nov. 15, 2023
(86) PCT No.: PCT/US2023/079919

§ 371 Date: May 14, 2025

Related U.S. Application Data
(60) Provisional application No. 63/425,990, filed on Nov. 16, 2022.

(51) Int. Cl. A61B 8/00; A61B 8/08

(52) U.S. Cl. CPC A61B 8/5223; A61B 8/0833; A61B 8/461; A61B 8/488

(57) ABSTRACT

In some embodiments, a computer-implemented method of monitoring health of a lung of a subject is provided. A computing device receives at least one signal from an ultrasound sensor. The computing device processes the at least one signal to detect an amount of a clinical sign of lung dysfunction. The computing device presents a metric based on the detected amount of the clinical sign of lung dysfunction.

### CROSS-REFERENCE TO RELATED APPLICATION

This application claims the benefit of Provisional Application No. 63/425,990, filed Nov. 16, 2022, the entire disclosure of which is hereby incorporated by reference herein for all purposes.

### STATEMENT OF GOVERNMENT LICENSE RIGHTS

This invention was made with government support under Grant No. R01 EB023910, awarded by the National Institutes of Health. The government has certain rights in the invention.

### BACKGROUND

Emergency Department (ED) patients who present with acute respiratory illness are triaged, diagnosed, and monitored for respiratory failure throughout hospitalization. Chest x-ray and CT are typically used for this purpose, but cannot be done continuously or serially and are associated with logistical limitations, for example when transporting unstable patients with hypoxemia or patients with respiratory infection due to the risk of contagion. Ultrasound is non-ionizing, rapid, accessible and has been shown to have high sensitivity for the diagnosis of pneumonia (including COVID-19), pulmonary edema, and ARDS, making ultrasound sensors suitable for triaging, diagnosing, and monitoring ED patients with acute respiratory illness.

Lung ultrasound (LUS) can also be used to monitor progression or improvement of disease and to adjust treatment regimen. For example, the presence, quality (focal vs diffuse), and number of LUS imaging artifacts—B-lines—are known to be correlated with the presence of fluid in the lung due to pneumonia, pulmonary edema, volume overload, fibrosis, pneumothorax, or other lung dysfunction. Per standard LUS protocol, scanning is performed in 6-10 anatomic zones to interrogate different locations of the lung, and the number and distribution (focal versus diffuse) of B-lines in each zone are determined. B-lines represent acoustic reverberations within regions of alveolar or interstitial edema adjacent to the pleural line. It is believed that B-lines correlate with the sizes of interlobular septa, but the exact mechanisms by which they form are still not fully understood.

While B-lines are correlated with the presence of lung dysfunction, one limitation of using LUS in this way is that the detection and quantification of B-lines requires specific training and is machine and operator dependent. While a number of recent studies on machine learning-based algorithms for automated B-line detection in ultrasound video output have a potential to address this problem, such LUS exams still utilize a multi-element US probe, an imaging system, and a skilled operator. Therefore, continuous, or frequent monitoring of lung condition through conventional LUS is logistically challenging. Further, in a number of clinical scenarios, it would be preferable to use adhesive wearable lung ultrasound sensors (LUSSes) that could be attached independently onto the anatomic zones of interest and interrogated on demand. In order to make such LUSSes miniature (approximate size similar to an EKG lead), the pulse-echo and signal processing electronics should be portable and affordable, which precludes the use of multi-element arrays and image reconstruction software.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a computer-implemented method of monitoring health of a lung of a subject is provided. A computing device receives at least one signal from an ultrasound sensor. The computing device processes the at least one signal to detect an amount of a clinical sign of lung dysfunction. The computing device presents a metric based on the detected amount of the clinical sign of lung dysfunction.

In some embodiments, a non-transitory computer-readable medium having computer-executable instructions stored thereon is provided. The instructions, in response to instruction by a computing system, cause the computing system to perform the method described above. In some embodiments, a computing system configured to perform the method described above is provided.

In some embodiments, a non-invasive system for monitoring lung health is provided. The system comprises a plurality of ultrasound sensors and a monitoring computing system. The ultrasound sensors are configured to be positioned on a thorax of a subject. The monitoring computing system is communicatively coupled to the plurality of ultrasound sensors and includes logic that, in response to execution by the monitoring computing system, causes the monitoring computing system to perform actions comprising: receiving ultrasound signals from the plurality of ultrasound sensors, and processing the ultrasound signals to generate a metric usable for clinical diagnosis.

### BRIEF DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1A is an example of an ultrasound image of a normally aerated lung.

FIG. 1B is an example of an ultrasound image of a lung exhibiting dysfunction.

FIG. 2 is a schematic illustration of a non-limiting example embodiment of a system that uses lung ultrasound sensors to monitor lung health of a subject, according to various aspects of the present disclosure.

FIG. 3 is an illustration of a non-limiting example embodiment of positions in which ultrasound sensors may be positioned in order to monitor lung health of a subject according to various aspects of the present disclosure.

FIG. 4A, FIG. 4B, and FIG. 4C illustrate a disassembled front view, an assembled front view, and a back view, respectively, of a non-limiting example embodiment of an ultrasound sensor according to various aspects of the present disclosure.

FIG. 5 is a block diagram that illustrates aspects of a non-limiting example embodiment of a monitoring computing system according to various aspects of the present disclosure.

FIG. 6 is a flowchart that illustrates a non-limiting example embodiment of a method of monitoring lung health of a subject according to various aspects of the present disclosure.

FIG. 7 is a flowchart that illustrates a non-limiting example embodiment of a procedure for detecting an amount of lung dysfunction based on an amount of pleural line reflectance variability according to various aspects of the present disclosure.

FIG. 8 is a flowchart that illustrates a non-limiting example embodiment of a procedure for detecting an amount of lung dysfunction based on a cumulative signal amplitude variability according to various aspects of the present disclosure.

FIG. 9 is a flowchart that illustrates a non-limiting example embodiment of a procedure for detecting an amount of lung dysfunction based on a cumulative signal amplitude variability and a pleural line reflectance variability according to various aspects of the present disclosure.

FIG. 10 is a flowchart that illustrates a non-limiting example embodiment of a procedure for detecting an amount of lung dysfunction based on a signal characteristic according to various aspects of the present disclosure.

FIG. 11 illustrates charts that show signal amplitude variability across multiple frames of video data according to a non-limiting experimental result related to the present disclosure.

FIG. 12A is a chart that illustrates a result of an initial cumulative analysis of candidate B-line indicators according to a non-limiting experimental result related to the present disclosure.

FIG. 12B is a chart that illustrates individual data points corresponding to each LUS dataset plotted in a ΔCA and DPₘₐₓ space according to a non-limiting experimental result related to the present disclosure.

### DETAILED DESCRIPTION

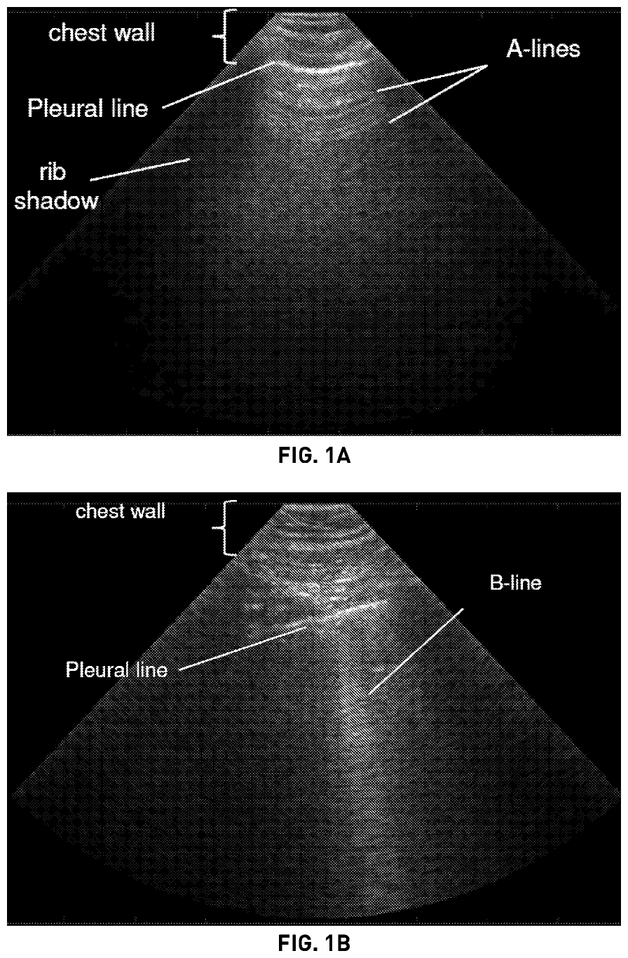

FIG. 1A is an example of an ultrasound image of a normally aerated lung. Between shadows of ribs, a bright pleural line is visible indicating a location of the pleura. A plurality of periodic horizontal lines parallel to the lung surface, known as A-lines, is also visible. The A-lines are imaging artifacts that, with lung sliding, indicate a normal aeration pattern. FIG. 1B is an example of an ultrasound image of a lung exhibiting dysfunction. The presence of B-lines-comet-like hyperechoic regions-indicate an alveolar or interstitial abnormality and stem from acoustic reverberations within regions of alveolar edema. The presence of B-lines is indicative of various types of lung dysfunction. For example, profuse bilateral B-lines with smooth pleural morphology are characteristic of cardiogenic pulmonary edema. As another example, focal B-lines with irregular pleural morphology are characteristic of pneumonia. Various other types of lung dysfunction, including but not limited to volume overload, may also be indicated by the presence of B-lines.

While the number and thickness of B-lines are correlated with a severity of lung dysfunction, visualization and quantification of B-lines requires substantial training, and even then is highly operator and machine dependent. The present disclosure provides wearable, automated, non-imaging lung ultrasound sensors (LUSSes) for continuous and automated monitoring of lung pathology while minimizing provider time, risk of virus exposure, and radiation. Individual LUSS elements may be attached to subjects in anatomic locations per existing standardized lung ultrasound diagnostic protocols, similarly to ECG leads. Raw ultrasound signals may be collected longitudinally and/or on demand. Signal processing techniques as described below may be used to extract quantitative metrics to evaluate lung edema severity and provide a simple metric that can be used in clinical decision making. In some embodiments, the metric may itself be used to automatically control one or more medical treatment devices.

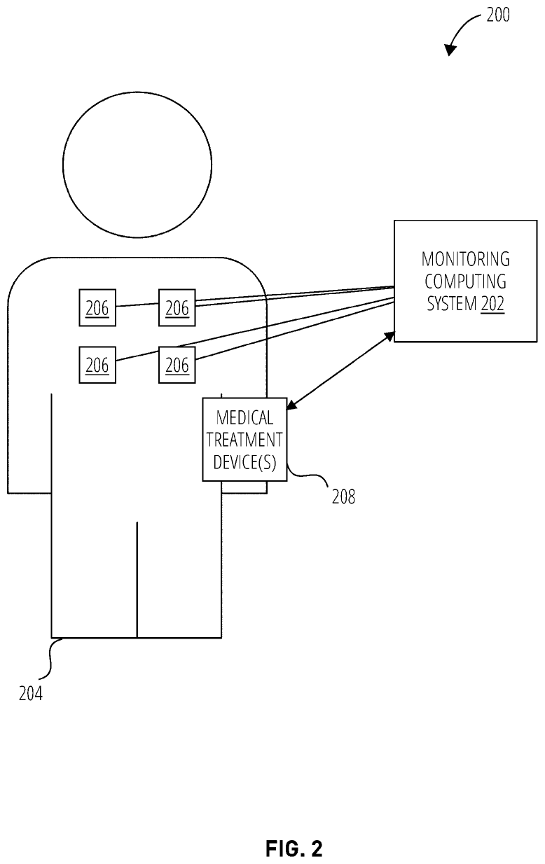

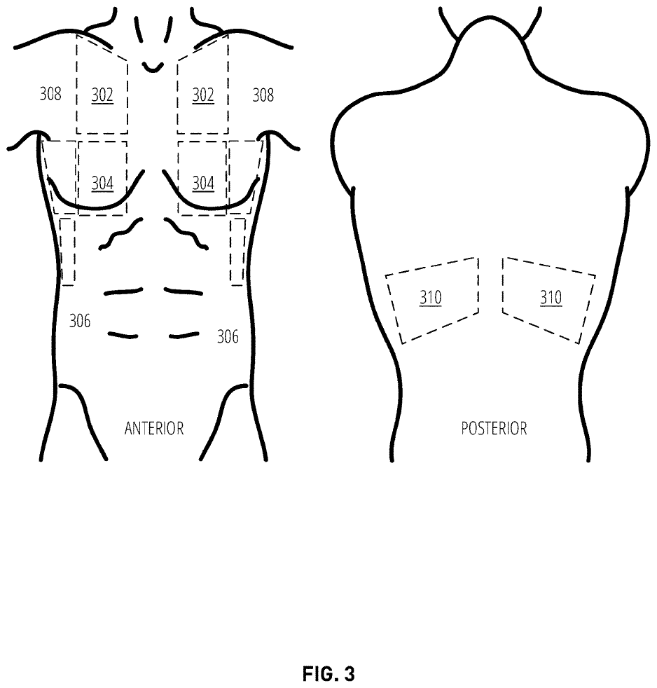

FIG. 2 is a schematic illustration of a non-limiting example embodiment of a system that uses lung ultrasound sensors to monitor lung health of a subject, according to various aspects of the present disclosure. In the system 200, a plurality of ultrasound sensors 206 are positioned on a subject 204 according to a diagnostic protocol, including but not limited to the 10-sensor protocol illustrated and described in FIG. 3.

Each of the ultrasound sensors 206 is communicatively coupled to a monitoring computing system 202 via a wired communication technology (e.g., coaxial cable, USB, Ethernet, or other suitable wired communication technology), wireless communication technology (e.g., Wi-Fi, Bluetooth, 5G, or other wireless communication technology), or any other suitable communication technology. The monitoring computing system 202 instructs the ultrasound sensors 206 to generate ultrasound to be applied to the subject 204, and receives signals sensed by the ultrasound sensors 206 in response.

In some embodiments, the monitoring computing system 202 is also communicatively coupled to one or more medical treatment devices 208. The monitoring computing system 202 may be communicatively coupled to any type of medical treatment device 208, including but not limited to one or more of a ventilator, an intravenous fluid dispenser, or a hemodialysis system. The medical treatment devices 208 may also include one or more sensors that do not themselves provide a treatment, including but not limited to a pulse oximeter, an electrocardiograph or a breath monitor. In some embodiments, the monitoring computing system 202 may use signals from the medical treatment devices 208 to gate signals received from the ultrasound sensors 206. In some embodiments, the monitoring computing system 202 may use determinations of lung dysfunction based on signals received from the ultrasound sensors 206 to determine control signals to be transmitted to the medical treatment devices 208. For example, if it is determined that a setting of a ventilator is causing or exacerbating lung dysfunction, the monitoring computing system 202 may transmit a command to change the setting of the ventilator.

FIG. 3 is an illustration of a non-limiting example embodiment of positions in which ultrasound sensors may be positioned in order to monitor lung health of a subject according to various aspects of the present disclosure. To monitor a given lung, ultrasound sensors 206 may be positioned in one or more of an infraclavicular region 302, a mammary region 304, an axilla 306, an upper axillary region 308, or an infrascapular region 310. In some embodiments, one ultrasound sensor 206 may be positioned on the subject 204 in each of these regions. In some embodiments, the ultrasound sensors 206 may be positioned bilaterally in order to monitor both lungs.

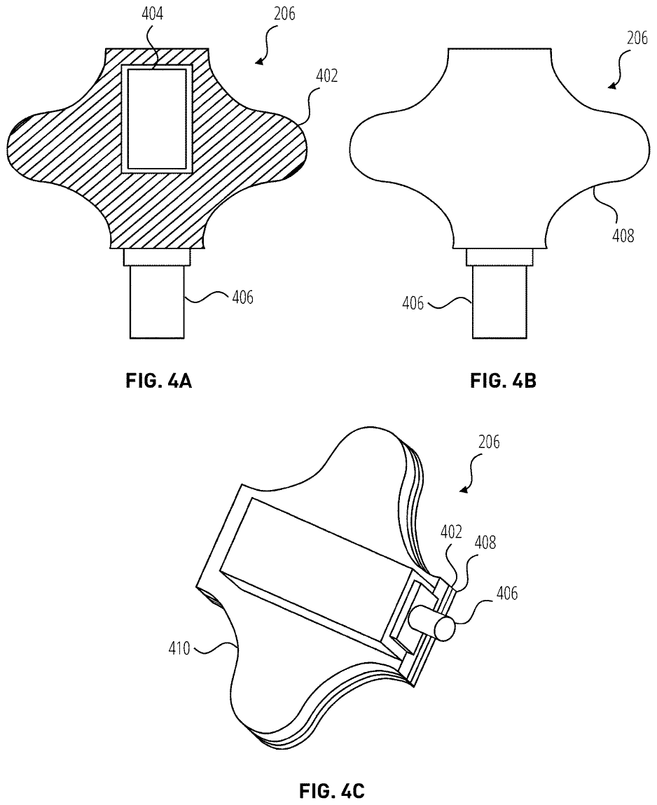

FIG. 4A, FIG. 4B, and FIG. 4C illustrate a disassembled front view, an assembled front view, and a back view, respectively, of a non-limiting example embodiment of an ultrasound sensor according to various aspects of the present disclosure. In the disassembled front view (FIG. 4A), a transceiver element 404 is shown, held in a housing 402. In some embodiments, the transceiver element 404 may include a piezoelectric ceramic transducer of a suitable type, including but not limited to an element conforming to a PZT Navy Type II standard. In some embodiments, the transceiver element 404 may have an area of 9 mm×5 mm, though in other embodiments, other sizes and/or shapes may be used. In some embodiments, the thickness of the transceiver element 404 may be one-half wavelength (2/2) of the acoustic energy it generates. A communication interface 406 is also illustrated. The illustrated communication interface 406 is a coaxial cable connection, though in other embodiments, other types of wired interfaces (including but not limited to direct soldered wiring, BNC connectors, or USB Type-C connectors) or wireless interfaces (including but not limited to Bluetooth) may be used. Though not illustrated, in some embodiments, a focusing lens may also be present.

In the assembled front view (FIG. 4B), a quarter wavelength matching layer 408 is applied over the housing 402 and the transceiver element 404. The thickness of the quarter wavelength matching layer 408 is one quarter wavelength (λ/4) of the acoustic energy generated by the transceiver element 404 in order to maximize transfer of acoustic energy into the subject 204. Any suitable material having an acoustic impedance that is between that of the skin of the subject 204 and the transceiver element 404 may be used. In some embodiments, a single quarter wavelength matching layer 408 is included. In some embodiments, more than one quarter wavelength matching layer 408 is included, such as two or more quarter wavelength matching layers 408.

In the back view (FIG. 4C), a lossy backing 410 is shown. The lossy backing 410 is a damping material whose presence improves performance of the transceiver element 404. The lossy backing 410 may be formed from any suitable material, including but not limited to an aluminum oxide-epoxy material. Though in the illustrated embodiment, the lossy backing 410, the housing 402, and the quarter wavelength matching layer 408 are illustrated in specific relative sizes, these sizes should not be seen as limiting, and in other embodiments, the lossy backing 410, housing 402, and/or quarter wavelength matching layer 408 may be different sizes than those illustrated in FIG. 4C. Further, as discussed above, even though a single quarter wavelength matching layer 408 is illustrated in FIG. 4C, in some embodiments, more than one quarter wavelength matching layer 408 is provided.

In use, an adhesive may be applied to a portion of the quarter wavelength matching layer 408, and the quarter wavelength matching layer 408 may be placed in contact with the skin of the subject 204 in a location as illustrated in FIG. 3. The communication interface 406 may be coupled to driving electronics. In some embodiments, the driving electronics may include a 40V pulse generator with a multiplex circuit that switches the pulses between two more ultrasound sensors 206 coupled to the driving electronics. The driving electronics may include transmit/receive switch circuits with +26 dB gain. In some embodiments, the driving electronics may be coupled to a monitoring computing system via wired (e.g., USB) or wireless (e.g., Bluetooth or WiFi) communication. In some embodiments, some portions of the driving electronics may be incorporated into the housing 402 of the ultrasound sensor 206 or the monitoring computing system. In some embodiments, the driving electronics may be included in their own housing separate from either the ultrasound sensors 206 or the monitoring computing system.

Though not illustrated, in some embodiments, an anatomic cue may be provided on the housing 402 of the ultrasound sensor 206 or elsewhere to guide where a particular ultrasound sensor 206 should be placed (e.g., in which of the zones illustrated in FIG. 3 a particular ultrasound sensor 206 should be placed, or a more detailed indicator of an anatomical feature to be aligned with the ultrasound sensor 206).

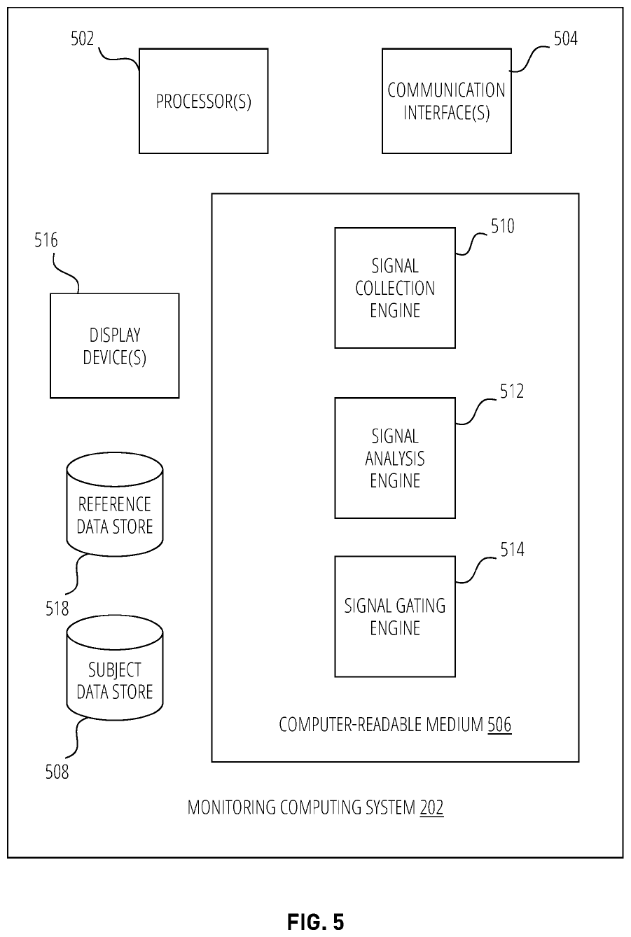

FIG. 5 is a block diagram that illustrates aspects of a non-limiting example embodiment of a monitoring computing system according to various aspects of the present disclosure. The illustrated monitoring computing system 202 may be implemented by any computing device or collection of computing devices, including but not limited to a desktop computing device, a laptop computing device, a mobile computing device, a server computing device, a computing device of a cloud computing system, and/or combinations thereof. As one non-limiting example, a first computing device of the monitoring computing system 202 may provide driving voltages to the ultrasound sensors 206 and receive analog return signals from the ultrasound sensors 206, perform an analog-to-digital conversion of the return signals, and provide the digital signals to a laptop computing device, a server computing device, or another computing device that provides the remaining components of the monitoring computing system 202. The monitoring computing system 202 is configured to use the ultrasound sensors 206 to sense characteristics of lung dysfunction of a subject 204, and to generate metrics representing an amount of sensed lung dysfunction. In some embodiments, the monitoring computing system 202 is also configured to control one or more medical treatment devices 208 based on the sensed amount of lung dysfunction.

As shown, the monitoring computing system 202 includes one or more processors 502, one or more communication interfaces 504, one or more display devices 516, a subject data store 508, a reference data store 518, and a computer-readable medium 506.

In some embodiments, the processors 502 may include any suitable type of general-purpose computer processor. In some embodiments, the processors 502 may include one or more special-purpose computer processors or AI accelerators optimized for specific computing tasks, including but not limited to graphical processing units (GPUs), vision processing units (VPTs), and tensor processing units (TPUs).

In some embodiments, the communication interfaces 504 include one or more hardware and or software interfaces suitable for providing communication links between components. The communication interfaces 504 may support one or more wired communication technologies (including but not limited to Ethernet, FireWire, and USB), one or more wireless communication technologies (including but not limited to Wi-Fi, WiMAX, Bluetooth, 2G, 3G, 4G, 5G, and LTE), and/or combinations thereof. In some embodiments, the communication interfaces 504 may include one or more wired or wireless interfaces for transmitting driving signals to the ultrasound sensors 206 and/or receiving signals detected by the ultrasound sensors 206.

In some embodiments, the display devices 516 may include one or more visual display devices (including but not limited to a monitor, touchscreen, indicator light, LCD display, or other type of visual display device), one or more audio display devices (including but not limited to a loudspeaker), and/or one or more hard-copy display devices (including but not limited to a printer).

As shown, the computer-readable medium 506 has stored thereon logic that, in response to execution by the one or more processors 502, cause the monitoring computing system 202 to provide a signal collection engine 510, a signal analysis engine 512, and a signal gating engine 514.

As used herein, “computer-readable medium” refers to a removable or nonremovable device that implements any technology capable of storing information in a volatile or non-volatile manner to be read by a processor of a computing device, including but not limited to: a hard drive; a flash memory; a solid state drive; random-access memory (RAM); read-only memory (ROM); a CD-ROM, a DVD, or other disk storage; a magnetic cassette; a magnetic tape; and a magnetic disk storage.

In some embodiments, the signal collection engine 510 is configured to cause driving signals to be transmitted to the ultrasound sensors 206 and to receive signals detected by the ultrasound sensors 206. In some embodiments, the signal gating engine 514 is configured to receive information from one or more medical treatment devices 208 that indicates when the signals collected by the signal collection engine 510 are appropriate for use by the signal analysis engine 512. In some embodiments, the signal analysis engine 512 is configured to use signals collected by the signal collection engine 510 (as gated by the signal gating engine 514) to measure amounts of lung dysfunction sensed by the ultrasound sensors 206, and to generate a metric indicating the measured amount of lung dysfunction. In some embodiments, the signal analysis engine 512 may cause the metric to be stored in the subject data store 508 for longitudinal analysis, may cause a display associated with the metric to be generated on one or more of the display devices 516, and/or may use the metric to determine a control signal to be transmitted to a medical treatment device 208. In some embodiments, the signal analysis engine 512 may use comparisons of signal characteristics of the signal to signal characteristics of previously collected signals stored in the reference data store 518 in order to determine the metric.

Further description of the configuration of each of these components is provided below.

As used herein, “engine” refers to logic embodied in hardware or software instructions, which can be written in one or more programming languages, including but not limited to C, C++, C#, COBOL, JAVA™, PHP, Perl, HTML, CSS, Javascript, VBScript, ASPX, Go, and Python. An engine may be compiled into executable programs or written in interpreted programming languages. Software engines may be callable from other engines or from themselves. Generally, the engines described herein refer to logical modules that can be merged with other engines, or can be divided into sub-engines. The engines can be implemented by logic stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device.

As used herein, “data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

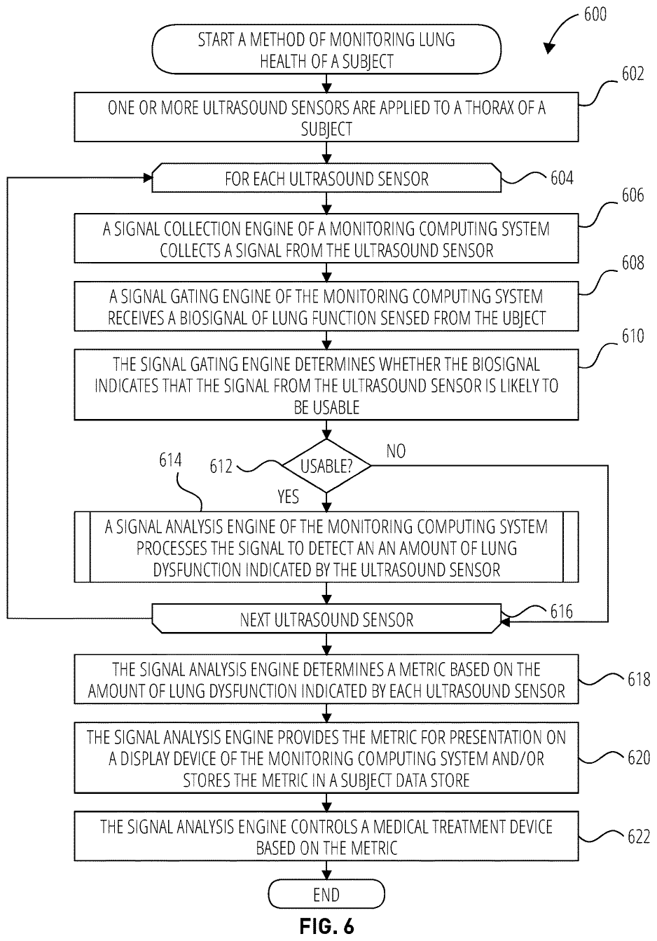

FIG. 6 is a flowchart that illustrates a non-limiting example embodiment of a method of monitoring lung health of a subject according to various aspects of the present disclosure. In the method 600, signals from one or more ultrasound sensors 206 are analyzed to detect signs of lung dysfunction, and a metric based on the analysis is presented to a clinician, stored for later use, or used to automatically control one or more medical treatment devices 208.

From a start block, the method 600 proceeds to block 602, where one or more ultrasound sensors 206 are applied to a thorax of a subject 204. The one or more ultrasound sensors 206 may be applied using any appropriate protocol. One protocol for lung ultrasound includes examining each lung in an infraclavicular region, a mammary region, an axilla, an upper axillary region, and an infrascapular region, as illustrated in FIG. 3. Accordingly, in some embodiments, one ultrasound sensor 206 may be applied to the subject 204 in each of these regions. In some embodiments, if both lungs are to be monitored, ultrasound sensors 206 may be applied to these regions bilaterally (e.g., one ultrasound sensor 206 in the region on the left side of the subject 204, and one ultrasound sensor 206 in the region on the right side of the subject 204), for a total of ten ultrasound sensors 206. In some embodiments, if only one lung is to be monitored or if only a limited region of the lung is to be monitored, fewer than ten ultrasound sensors 206 may be used. In some embodiments, more than one ultrasound sensor 206 may be applied to one or more of the regions.

The method 600 then proceeds to a for-loop defined between a for-loop start block 604 and a for-loop end block 616, wherein signals from each of the one or more ultrasound sensors 206 is processed. In some embodiments, each of the ultrasound sensors 206 may be processed serially (e.g., the entire for-loop is executed for a first ultrasound sensor 206 before executing the entire for-loop for a subsequent sensor). In some embodiments, at least two of the ultrasound sensors 206 may be processed in parallel (e.g., at least a portion of the for-loop for two ultrasound sensors 206 may be executed concurrently).

From the for-loop start block 604, the method 600 proceeds to block 606, where a signal collection engine 510 of the monitoring computing system 202 collects a signal from the ultrasound sensor 206. In some embodiments, the driving electronics may provide a voltage to the ultrasound sensor 206 to generate one or more pulses, and the signal may be an analog return detected by the ultrasound sensor 206. In some embodiments, the signal may be a processed version of the output of the ultrasound sensor 206, such as an array including a plurality of values indicating a strength of a return signal at a plurality of distances from the ultrasound sensor 206. In some embodiments, the signal collected may represent a single point in time. In some embodiments, the signal collected may include a time series of values collected over time.

In some embodiments, the signal from the ultrasound sensor 206 reliably indicates a presence of lung dysfunction during certain portions of a respiration cycle (i.e., cycles of inhalation and exhalation), but does not necessarily reliably indicate the presence of the lung dysfunction during other portions of the respiration cycle. For example, in some embodiments it may be desirable to use signals from a point in the respiration cycle when the lung is fully expanded or fully contracted, such that the lung is relatively motionless. As another example, in some embodiments, it may be desirable to use signals that cover an entire breath-to-breath timeframe. Accordingly, at block 608, a signal gating engine 514 of the monitoring computing system 202 receives a biosignal of lung function sensed from the subject 204, and at block 610, the signal gating engine 514 determines whether the biosignal indicates that the signal from the ultrasound sensor 206 is likely to be usable. In some embodiments, the biosignal may be received from a medical treatment device 208, and may be indicative of a respiration cycle of the subject 204. Some non-limiting examples of suitable biosignals include a respiratory rate, an oxygen level, a pressure-volume trace, or an end-tidal carbon dioxide level.

At decision block 612, a determination is made based on whether the signal is likely to be usable (i.e., is from the desired portion of the respiration cycle). If the signal is not likely to be usable, then the result of decision block 612 is NO, and the method 600 advances to the for-loop end block 616. Otherwise, if the signal is likely to be usable, then the result of decision block 612 is YES, and the method 600 advances to procedure block 614. Though the signal gating is described in blocks 608 and 610, in some embodiments, signal gating may not be used, and all signals collected are processed. Further, though the method 600 is illustrated as discarding signals from a sensor if the signal gating engine 514 determines that they are unlikely to be usable, in some embodiments, the method 600 may instead loop back to block 606 to collect a new signal from the ultrasound sensor 206 until a signal likely to be usable is obtained.

At procedure block 614, a procedure is executed wherein a signal analysis engine 512 of the monitoring computing system 202 processes the signal to detect an amount of lung dysfunction indicated by the ultrasound sensor 206. The amount of lung dysfunction may be specified in any suitable manner. For example, the procedure may determine segments of the signal that indicate lung dysfunction and segments of the signal that do not indicate lung dysfunction, and provide the amount as the percentage of the signal that does or does not indicate lung dysfunction. As another example, the procedure may return a value that indicates whether or any lung dysfunction is indicated by the signal. As yet another example, the procedure may return a value that indicates a level of lung dysfunction indicated by the signal (e.g., none, mild, moderate, severe), based on thresholds (e.g., <1%, 1-10%, 11-25%, >25%) or other characteristics of its analysis.

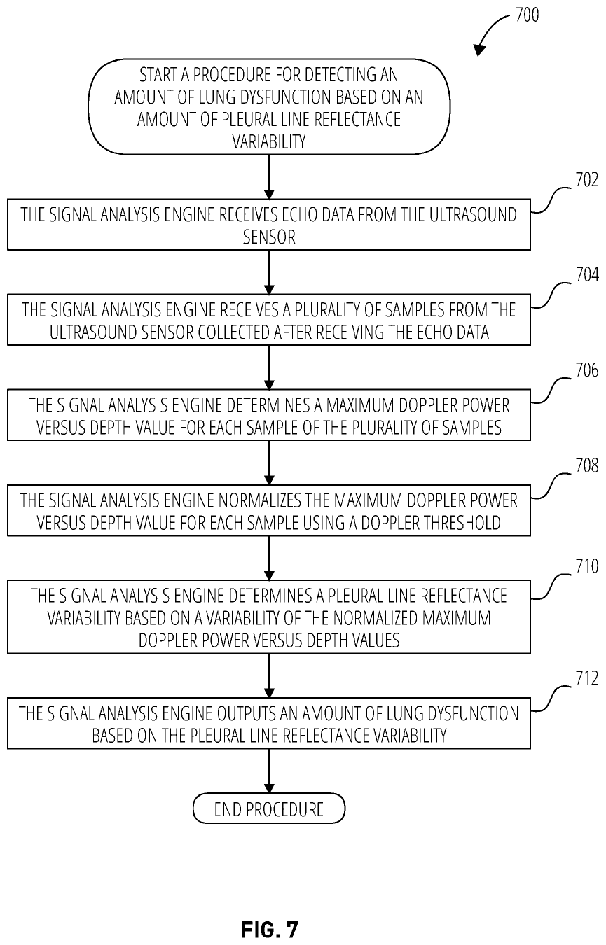

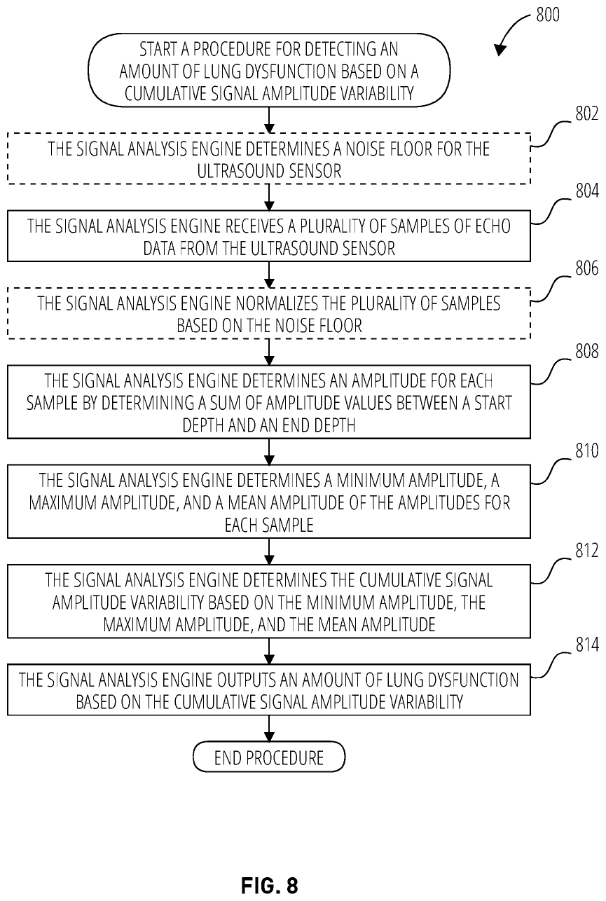

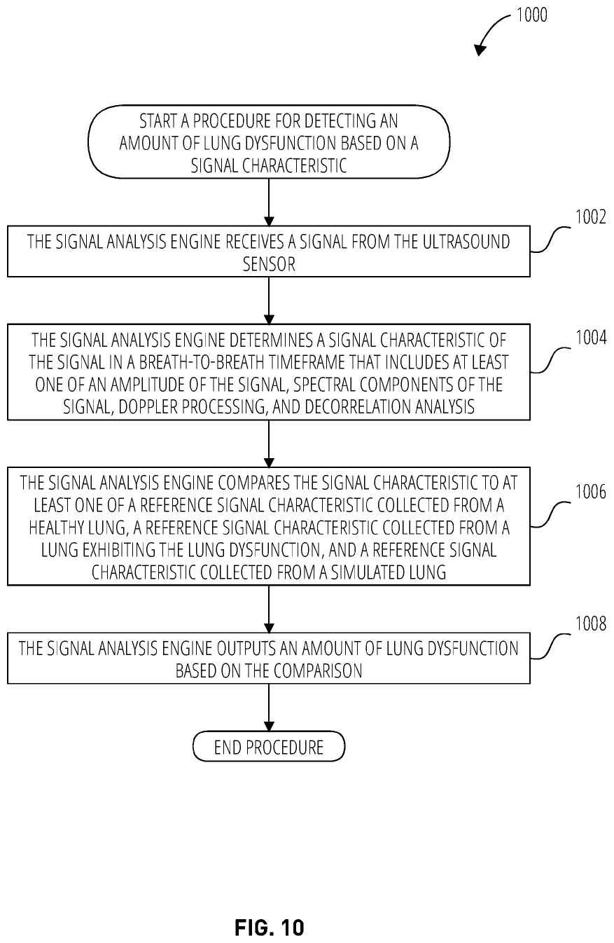

Any suitable technique for determining the amount of lung dysfunction may be used, including techniques that use analysis of the raw signal from the ultrasound sensor 206 that do not require the generation of an image. Raw signals tend to have a greater dynamic range and therefore more sensitivity, and can be processed more quickly with simpler instrumentation than if an image is generated. Several non-limiting examples of such techniques are illustrated in FIG. 7, FIG. 8, and FIG. 10, and are discussed in further detail below.

The method 600 then advances to the for-loop end block 616. At for-loop end block 616, if further ultrasound sensors 206 remain to be processed, then the method 600 returns from the for-loop end block 616 to the for-loop start block 604 to process the next ultrasound sensor 206. Otherwise, if all of the ultrasound sensors 206 have been processed, then the method 600 advances from the for-loop end block 616 to block 618.

At block 618, the signal analysis engine 512 determines a metric based on the amount of lung dysfunction indicated by each ultrasound sensor 206. In some embodiments, the signal analysis engine 512 may combine the amounts of lung dysfunction indicated by each of the ultrasound sensors 206 to determine a total amount of lung dysfunction to be used as the metric. In some embodiments, the signal analysis engine 512 may average the amounts of lung dysfunction indicated by each of the ultrasound sensor 206 to determine an average amount of lung dysfunction to be used as the metric. In some embodiments, the signal analysis engine 512 may use the maximum amount of lung dysfunction indicated by any of the ultrasound sensors 206 as the metric. In some embodiments, the signal analysis engine 512 may use a number or percentage of ultrasound sensors 206, or number/percentage of monitoring zones that indicate any non-zero level of lung dysfunction as the metric (e.g., None if ≥8 zones indicate no lung dysfunction, Mild if >8 zones indicate Mild or no lung dysfunction, Moderate if 3-5 zones indicate Moderate lung dysfunction and ≤4 zones indicate Severe lung dysfunction, Severe if >5 zones indicate Severe lung dysfunction). In other embodiments, the metric may be determined based on the amounts of lung dysfunction indicated by the ultrasound sensors 206 in any other suitable way.

At block 620, the signal analysis engine 512 provides the metric for presentation on a display device 516 of the monitoring computing system 202 and/or stores the metric in a subject data store 508. In some embodiments, a numerical, textual, iconic, or other direct representation of the metric may be presented. In some embodiments, if the metric indicates an amount of lung dysfunction greater than a threshold, providing the metric for presentation may include presenting an alarm. Storing the metric in a subject data store 508 may allow longitudinal reports for the subject 204 to be generated and/or compared to longitudinal data for other subjects.

At block 622, the signal analysis engine 512 controls a medical treatment device 208 based on the metric. Since some medical treatment devices 208 may cause or exacerbate lung dysfunction if not adjusted, using the metric to automatically adjust operational settings of such medical treatment devices 208 can greatly improve care. For example, operational settings of a ventilator, an intravenous fluid dispenser, or a hemodialysis system may be adjusted based on the metric per standard protocols for adjusting the medical treatment devices 208 in the presence of the detected lung dysfunction.

Though the method 600 is illustrated and described as performing each of the actions in block 620 and block 622, in some embodiments, the method 600 may perform one or more of the presentation of the metric, storage of the metric, and control of the medical treatment device 208 without performing all three.

The method 600 then proceeds to an end block and terminates. Though illustrated as terminating for the ease of discussion, the method 600 may loop back to for-loop start block 604 to continue to monitor the subject 204 over time.

While any suitable technique may be used at procedure block 614, it has not been previously known how to detect B-lines or other signals of lung dysfunction from raw signals produced by an LUSS such as ultrasound sensor 206. One non-limiting example technique for such detection is to analyze pleural line reflectance variability, or maximum Doppler power. Pleural line reflectance variability may be determined by comparing a sequence of multiple (e.g., 33 in a non-limiting example) identical ultrasound pulses reflected from the pleural line. In some embodiments, those pulses may be collected following the collection of echo data (such as, but not limited to, following a B-mode image or signals usable to create a B-mode image so that each set of signals usable to create a B-mode image has a corresponding set of pulses).

If the pleural line is homogenously reflecting (as in the case of a fully aerated lung), each of the reflections from the pleural line will be very similar, and the differences between them will be minimal (e.g., at the noise floor). If there is a defect on the pleural line, such as an area of fibrosis or an area of edema in contact with the pleural line, such defects represent a trap for the ultrasound pulses that result in the formation of B-lines. If such a defect is moving with respiration or heart beat relatively to the ultrasound beam generated by the ultrasound sensor 206, then the reflections will not be the same for all of the pulses. By quantifying the differences between the reflections, a value that is correlated with the presence of B-lines, and therefore with the presence of lung dysfunction, can be determined.

Doppler-like processing is one way to quantify the differences between the reflections. Doppler power is based on a sum of magnitudes of differences between each consecutive pair of pulses. Thus, a set of pulses will yield a single signal for Doppler power vs depth. A maximum of that signal is co-located with the pleural line, because the membranes that form pleura slide relative to each other during breathing, and are therefore expected to move relative to the ultrasound sensor 206 during breathing. This maximum signal, normalized by a noise floor for a corresponding set of echo data and/or a Doppler threshold, constitutes a pleural line reflectance value. The variability between the pleural line reflectance values may then be considered the pleural line reflectance variability, and may be provided as a value that indicates an amount of lung dysfunction. This has been found to be a sensitive indicator of B-lines that move with respiration.

FIG. 7 is a flowchart that illustrates a non-limiting example embodiment of a procedure for detecting an amount of lung dysfunction based on an amount of pleural line reflectance variability according to various aspects of the present disclosure. The procedure 700 assumes that the signal collected at, for example, block 606, includes at least one set of echo data and a corresponding set of pulses as described above.

From a start block, the procedure 700 advances to block 702, where the signal analysis engine 512 receives echo data from the ultrasound sensor 206. In some embodiments, the echo data is B-mode data, which has values that vary based on the amplitude of the received signal.

At block 704, the signal analysis engine 512 receives a plurality of samples from the ultrasound sensor 206 collected after receiving the echo data. As discussed above, the plurality of samples may include samples from a set of pulses (e.g., 33 pulses) transmitted after the collection of the echo data.

At block 706, the signal analysis engine 512 determines a maximum Doppler power versus depth value for each sample of the plurality of samples. In some embodiments, each sample includes an array of Doppler power values, with each element in the array indicating a depth away from the ultrasound sensor 206. The maximum Doppler power versus depth value coincides with the location of the pleural line, and so block 706 finds the Doppler power versus depth value that coincides with the pleural line.

At block 708, the signal analysis engine 512 normalizes the maximum Doppler power versus depth value for each sample using a Doppler threshold. This normalization may include determining the Doppler threshold by averaging Doppler power over the entire depth range, and multiplying the average Doppler power by two, and then using this Doppler threshold to normalize the maximum Doppler power versus depth values.

At block 710, the signal analysis engine 512 determines a pleural line reflectance variability based on a variability of the normalized maximum Doppler power versus depth values. The variability may be determined using any suitable technique, including but not limited to determining a range of the values, an interquartile range of the values, a standard deviation of the values, a variance of the values, or any other suitable technique for representing variability.

At block 712, the signal analysis engine 512 outputs an amount of lung dysfunction based on the pleural line reflectance variability. In some embodiments, the variability determined at block 710 is itself output as the amount of lung dysfunction.

The procedure 700 then advances to an end block and returns control to its caller.

While the pleural line reflectance variability is a useful indicator, in some embodiments, it may be too sensitive to use as a sole indicator of lung dysfunction such as pulmonary edema, as it may pick up imperfections in the pleural line that do not form proper B-lines, but instead form small short comet tails (sometimes referred to as C-lines), if that. Accordingly, in some embodiments, additional indicators may be used in addition to or instead of the pleural line reflectance variability. One such indicator is a cumulative signal amplitude variability. This indicator leverages the definition of B-line in that B-lines extend all the way down to a 12 cm depth. Theoretically, an absolute cumulative brightness (or signal amplitude) over an area at a large depth (e.g., 9-12 cm) to distinguish B-lines from non-B-lines. However, this runs the risk of mistaking A-lines or other reverberation signals for a B-line. Further, the absolute brightness depends on habitus of the body of the subject 204. Accordingly, a transient, “shimmering” quality of B-lines at large depth, expressed as brightness variability across sets of echo data (e.g., data usable to create a plurality of B-mode frames), may be used to separate out false positives.

FIG. 8 is a flowchart that illustrates a non-limiting example embodiment of a procedure for detecting an amount of lung dysfunction based on a cumulative signal amplitude variability according to various aspects of the present disclosure. The procedure 800 assumes that the signal collected at, for example, block 606, includes a plurality of sets of echo data as described above.

From a start block, the procedure 800 advances to optional block 802, where the signal analysis engine 512 determines a noise floor for the ultrasound sensor 206. In some embodiments, the noise floor may be determined by receiving echo data from the ultrasound sensor 206 prior to the ultrasound sensor 206 being applied to the subject 204 (e.g., in the air). This echo data will include only electronic noise because a return signal will not be received, and so the amplitude of this electronic noise may be considered the noise floor.

At block 804, the signal analysis engine 512 receives a plurality of samples of echo data from the ultrasound sensor 206, and at optional block 806, the signal analysis engine 512 normalizes the plurality of samples based on the noise floor. Each sample of echo data may be a type of data suitable to create a B-mode image, though creation of an image is not necessary.

At block 808, the signal analysis engine 512 determines an amplitude for each sample by determining a sum of amplitude values between a start depth and an end depth. As discussed above, the echo data of each sample may include an array of values, wherein each element in the array indicates a value at a specific depth. As such, the signal analysis engine 512 may sum the amplitude values at the desired positions in the array to determine the sum of amplitude values. Any suitable start depth and end depth may be used. In some embodiments, a start depth of 9 cm and an end depth of 12 cm may be used. These values are non-limiting examples only, and in some embodiments, the start depth may be chosen from a range of 8 cm-10 cm, and the end depth may be chosen from a range of 11 cm-13 cm.

At block 810, the signal analysis engine 512 determines a minimum amplitude, a maximum amplitude, and a mean amplitude of the amplitudes for each sample, and at block 812, the signal analysis engine 512 determines the cumulative signal amplitude variability based on the minimum amplitude, the maximum amplitude, and the mean amplitude. Any suitable technique may be used to convert the minimum amplitude, maximum amplitude, and mean amplitude to the cumulative signal amplitude variability. One non-limiting example of a conversion is use of a function such as:

$$Δ {A}_{D} = \frac{{A}_{min} - {A}_{max}}{{A}_{mean}}$$

wherein ΔA_{D} is the signal amplitude variability for a given sample, Aₘᵢₙ is the minimum amplitude for the given sample, Amax is the maximum amplitude for the given sample, and Aₘₑₐₙ is the mean amplitude for the given sample. The signal amplitude variabilities ΔA_{D} for each of the samples may be combined to determine the cumulative signal amplitude variability.

At block 814, the signal analysis engine 512 outputs an amount of lung dysfunction based on the cumulative signal amplitude variability. In some embodiments, the cumulative signal amplitude variability itself may be output as the amount of lung dysfunction.

The procedure 800 then advances to an end block and returns control to its caller.

In some embodiments, determining the noise floor at optional block 802 and using it to normalize the samples at optional block 806 may help improve the accuracy of the determination of the cumulative signal amplitude variability. In some embodiments, optional block 802 and optional block 806 may be skipped, since subsequent processing of the values may render any normalization redundant. For example, embodiments that divide by the mean amplitude for the given sample as described with respect to the function above may skip optional block 802 and optional block 806 without affecting the accuracy of the determination of the cumulative signal amplitude variability.

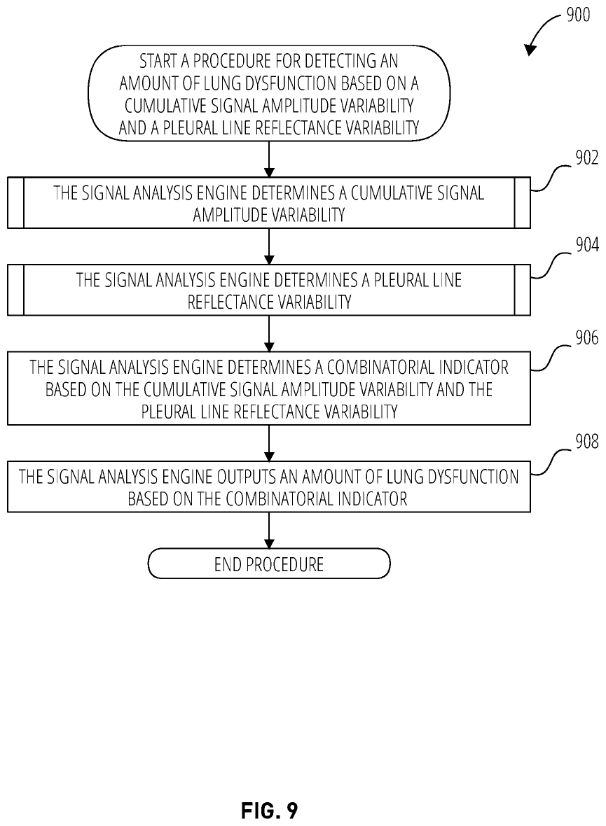

In addition to determining individual indicators of lung dysfunction, in some embodiments, multiple indicators may be used in combination to increase the reliability of the determination. FIG. 9 is a flowchart that illustrates a non-limiting example embodiment of a procedure for detecting an amount of lung dysfunction based on a cumulative signal amplitude variability and a pleural line reflectance variability according to various aspects of the present disclosure.

From a start block, the procedure 900 advances to procedure block 902, where a procedure is executed wherein the signal analysis engine 512 determines a cumulative signal amplitude variability. Any suitable technique for determining the cumulative signal amplitude variability, including but not limited to the techniques of the procedure 800 illustrated in FIG. 8, may be used.

At procedure block 904, a procedure is executed wherein the signal analysis engine 512 determines a pleural line reflectance variability. Any suitable technique for determining the pleural line reflectance variability, including but not limited to the techniques of the procedure 700 illustrated in FIG. 7, may be used.

At block 906, the signal analysis engine 512 determines a combinatorial indicator based on the cumulative signal amplitude variability and the pleural line reflectance variability. In some embodiments, each value may be multiplied by a coefficient determined via regression analysis to create the combinatorial indicator.

At block 908, the signal analysis engine 512 outputs an amount of lung dysfunction based on the combinatorial indicator. In some embodiments, the combinatorial indicator may be compared to a detection threshold, and the presence or absence of a B-line (i.e., an indication of the presence of lung dysfunction) may be output if the combinatorial indicator is greater than the detection threshold.

The procedure 900 then advances to an end block and returns control to its caller.

In some embodiments, instead of calculating a specific indicator of lung dysfunction such as pleural line reflectance variability or cumulative signal amplitude variability, an amount of lung dysfunction may be determined by comparing characteristics of a signal detected by an ultrasound sensor 206 to sample signals previously collected from healthy lungs, lungs exhibiting the lung dysfunction, and/or artificial simulations. FIG. 10 is a flowchart that illustrates a non-limiting example embodiment of a procedure for detecting an amount of lung dysfunction based on a signal characteristic according to various aspects of the present disclosure.

From a start block, the procedure 1000 advances to block 1002, where the signal analysis engine 512 receives a signal from the ultrasound sensor 206. The signal received may be echo data, pulse data, or any other type of signal generated by the ultrasound sensor 206 that matches the reference data previously collected for comparison.

At block 1004, the signal analysis engine 512 determines a signal characteristic of the signal in a breath-to-breath timeframe that includes at least one of an amplitude of the signal, spectral components of the signal, a result of Doppler processing, and a result of decorrelation analysis. In some embodiments, the signal gating engine 514 may have detected the breath-to-breath timeframe and limited the signal provided to the block 1002 to signals from that desired period.

At block 1006, the signal analysis engine 512 compares the signal characteristic to at least one of a reference signal characteristic collected from a healthy lung, a reference signal characteristic collected from a lung exhibiting the lung dysfunction, and a reference signal characteristic collected from a simulated lung. In some embodiments, the reference signal characteristic may be stored in a reference data store 518 of the monitoring computing system 202 for the comparison.

At block 1008, the signal analysis engine 512 outputs an amount of lung dysfunction based on the comparison. In some embodiments, the signal analysis engine 512 may determine whether the signal characteristic is more like the reference signal characteristic from the lung exhibiting the lung dysfunction or one of the other reference signal characteristics, and may determine an amount of lung dysfunction based on the comparison.

The procedure 1000 then advances to an end block and returns control to its caller.

### Experimental Results

A prospective observational cohort study was conducted at the University of Washington (UW) Medical Center to verify the effectiveness of non-limiting example embodiments of the above-disclosed techniques. Patients admitted to the hospital with a presumed diagnosis of cardiogenic pulmonary edema were eligible for enrollment, with target enrollment of n=15. Five healthy volunteers were also enrolled from the study team. Excluded were participants who were less than 18 years of age, incarcerated, pregnant, unable to provide consent in English, had known COVID infection, or patients whose condition required acute intervention such as urgent angiography or emergency mechanical device implantation for cardiogenic shock.

Each participant received a LUS exam per a standard 10-zone protocol as illustrated in FIG. 4A-FIG. 4B performed by a certified sonographer or an attending emergency physician trained in lung ultrasound. The protocol consisted of positioning an US transducer (64-element phased array S4-2M, Humanscan, Seoul, South Korea) consecutively over each zone, with the imaging plane perpendicular to the intercostal spaces. With the probe immobile and the subject respirating freely, a 3-second cine loop of B-mode ultrasound was recorded at 15 frames per second (fps), resulting in a 45-frame video per lung zone. The US probe was operated by an open platform ultrasound system-Verasonics Ultrasound Engine V1 (Verasonics, Kirkland, WA, USA) with imaging settings replicating those of the lung preset of Mindray TE7 (Mindray Medical International, Shenzen, China): operating frequency 4.5 MHz, sector angle 45°, number of scan lines 128, focusing depth 2.5 cm, depth 12 cm, MI 0.8.

During the recording of LUS video in each zone, the echoes received by all elements of the array (referred to as radiofrequency (RF) signals) in each frame corresponding to all the scan lines (128 per frame) were recorded at the sampling frequency of 20 MHz. Each set of 128 scan lines forming a LUS image frame was followed by 35 additional US pulses emitted without focusing (i.e., as plane waves in a wide beam) at pulse repetition frequency (PRF) of 5 kHz, and the corresponding RF signals were also recorded at the same sampling frequency and analyzed in post-processing as described below.

Three emergency department physicians trained in LUS and blinded to the subject cohort (but not the zone number) independently reviewed the LUS videos and provided a binary score indicating presence or absence of B-lines. Cohen's Kappa was calculated to assess the degree of agreement between two expert clinical reviewers, and a third expert clinical reviewer served as a tiebreaker in cases with discordant image interpretations.

RF signal analysis was performed independently of the corresponding LUS video review. The following groups of RF signal metrics were considered as candidates for B-line indicators: signal amplitude at large (8-12 cm) depth, frequency spectrum of the signal, and signal phase and amplitude temporal variability. All of the indicators were computed for each RF signal set corresponding to a single frame in a LUS video, and then maximum, minimum, and average over the 45 frames were calculated. The rationale behind selecting those groups of indicators was based on qualities of B-lines known from the literature and observed in practice, and the hypothesized mechanisms of B-line formation. Specifically, RF signal amplitude at a certain time point determines the brightness of the US image at the corresponding spatial location. Because B-lines are bright artifacts, it is reasonable to hypothesize that they will correspond to RF signals with large amplitude. Furthermore, B-lines by definition extend from the position of the pleural line to at least 12 cm depth, where no other bright reflectors are expected. To account for differences in body habitus and thus depth of pleural line location, the range of RF signal arrival time, t, and corresponding depth, d=c₀t/2, where c₀ is the speed of sound, was selected as 8-12 cm for signal amplitude analysis.

First, cumulative RF signal S(d) was obtained by summation of RF signals s(d) over the central 16 elements of the US probe (for both focused and wide beam acquisitions) and over 32 central scan lines (for the focused acquisitions). Cumulative signal amplitude for both wide beam plane wave and focused beam acquisitions, CA_{W} and CA_{F} respectively, was then calculated by integration of the absolute value of S(d) over depth within the 8-12 cm range. Both of those indicators were then normalized by their corresponding noise values calculated from reference blank LUS acquisitions, where the probe was not in contact with a subject. Mean and maximum CA indicators were computed over 45 frames of each video.

Analyzing the temporal variability of RF signal amplitude at the time scale corresponding to the frame rate of LUS was motivated by the “shimmering” quality of B-lines relatively to echo texture of soft tissues, reverberations, and clutter that may be encountered in LUS. Thus, cumulative signal amplitude variability, ΔCA_{F}, was calculated for focused beam RF datasets corresponding to a video clip as follows:

ΔCA_{F}=(CA_{Fmax}−CA_{Fmin})·100%/CA_{Fmean}
where CA_{Fmax}, CA_{Fmin} and CA_{Fmean} represent maximum, minimum, and mean values of CA_{F} over the 45 frames.

Analysis of the temporal variability of the RF signal at the shorter time scale-within milliseconds—was motivated by the mechanism hypothesized in the literature to be responsible for B-line formation—reverberation of the LUS imaging pulses within irregularly-shaped fluid-filled areas of the lung adjacent to the pleural line. The dynamics of this reverberation determines the shape of the RF signal over its arrival time, and is expected to be sensitive to the relative LUS beam position and angle of incidence that both change with physiological motion. Therefore, we hypothesized that RF signal corresponding to a B-line may be highly variable over short-millisecond-time scales, i.e., from one wide beam plane wave pulse to the next. The variability was quantified by Doppler-like processing of the 35 consecutive RF signals from plane wave acquisitions. The RF signals on the central 16 elements were summed, transformed into in-phase and quadrature (I/Q) components through Hilbert transform, and wall filtered by linear regression. Doppler power distribution over depth DP(d) was then computed as autocorrelation with a lag of 1 per Kasai et al. Doppler threshold was set as the average of DP(d) multiplied by a factor of two, and the maximum value of DP(d) divided by this threshold was the output indicator for this frame, DP. Mean and maximum DP were computed over 45 frames of each video.

Lastly, frequency spectra of the RF signals beamformed and summed over the central 16 elements were computed for a limited number (n=10) of central scan lines that contained well defined B-lines and compared to those that did not contain it. This limited investigation was motivated by a hypothesis posed in a numerical study that the signal frequency spectrum could be indicative of the native frequencies of acoustic traps—fluid-filled areas in the lung—and would thus be informative and indicative of B-lines. However, this limited initial analysis did not reveal any consistent changes in the spectral width or shape associated with B-lines, consistently with Kameda et al, and thus further analysis was not pursued.

All of the candidate indicators of B-lines were calculated for all LUS datasets and assigned to two groups—B-lines present or absent—according to the binary outcome of the expert review. Two indicators with the highest level of statistical significance (per two-sided Student's t-test) and the largest effect size were selected for further analysis of predictive accuracy.

The binary evaluation of the LUS videos by independent reviewers described above (B-line present/absent) served as the ground truth for evaluating predictive accuracy of the candidate signal indicators. The relationship between the two candidate signal indicators and presence of B-lines was modeled via logistic regression. Predictive performance of the regression model was assessed via k-fold cross-validation (k=10), whereby the total dataset was randomly divided into k groups. The first test data set (used to test the accuracy of the predictive model) was set aside and the remaining (k−1) groups served as the training data set (used to fit the predictive logistic regression model). The process was repeated until all k groups had been used in turn as test and training datasets. Receiver Operator Characteristics (ROC) analysis was used to quantify overall performance of the candidate indicators in terms of cross-validated area under the curve (cvAUC; stata package cvauroc). cvAUC was calculated by averaging AUCs from each fold and applying bootstrapping procedures to calculate 95% confidence intervals. Contingency tables were also generated to report classifications and evaluate diagnostic accuracy, sensitivity, specificity, positive predictive value, and negative predictive value. Stata 15.1 (College Station, TX) was used for all statistical analyses.

From May to November 2021, 157 participants were screened, 29 were eligible, and 12 were enrolled with complete imaging. Reasons for non-enrollment of eligible participants included staff being unavailable (9), participants not consenting (4) or other (3). Ultimately 12 patients and 5 healthy volunteers were scanned using the 10-zone LUS protocol; the video clips were not collected for one healthy volunteer due to a technical issue with data recording, and thus were not included in the study. Patients enrolled in the study were admitted from the emergency department for a presumptive diagnosis of acute decompensated heart failure, with all 12 having chest radiograph findings consistent with acute pulmonary edema.

Overall, 160 individual LUS video clips were recorded and scored by 2 blinded reviewers for the presence or absence of B-lines. Inter-rater agreement was 89%, kappa 0.79 (95% CI=0.63-0.94). Discordant interpretations were adjudicated by consensus with the third expert reviewer in 10.2% of cases, resulting in 77 and 83 video clips with B-lines absent and present, respectively. Complete sets of quality RF signals were collected for 114 video clips, of which 41 and 73 corresponded to the LUS videos with B-lines absent and present, respectively. Those datasets were included in the statistical analysis of candidate B-line indicators; the excluded datasets were either incomplete (n=30 from three healthy volunteers and n=6 from patients, due to data recording issues) or contained noisy RF data (n=10 from a patient with very thick chest wall) such that not all of the candidate metrics could be computed.

B-lines formed in LUS images in the case of wide beam acquisitions, albeit with diminished contrast vs focused scan line images. This is contrary to previously reported expectations that a narrow LUS beam is required for trapping of LUS pulses and B-line formation. The brightness of B-line in LUS images corresponded to higher RF signal amplitude at large depth for both focused scan lines and plane wave acquisitions, although in the latter case the RF signal dropped below the noise floor at depths beyond 10 cm. The lower RF signal amplitude was to be expected, as the pressure in the wide LUS beam incident onto the lung surface was much lower than in the focused scan lines. To account for this, the depth range of 6-10 cm was chosen for batch analysis of cumulative amplitude CA_{W} in wide beam RF signals.

LUS dataset examples illustrated the temporal variability of the RF signal. Variability at the short time scales, described by DP(z), typically had low amplitude peaks, often below noise floor, within the depth range corresponding to the chest wall, as expected for perfused tissue. In datasets without B-lines, DP(z) was most commonly at the noise floor throughout depth, with occasional small amplitude peaks within the chest wall and/or at the pleural line. A much higher amplitude peak was consistently observed at the position of the pleural line only in the cases when B-lines were present. The amplitude of the peak was qualitatively correlated with the number and brightness of B-lines, as well as the amount of their motion with respiration. In most cases this peak was narrow and confined to the location of the pleural line, with DP(d) returning to the noise floor at larger depths. However, there were instances, typically associated with very bright, pronounced B-lines, where the peak was wider and extended up to 2 cm depth below the pleural line.

The observations above and analysis of the corresponding sets of RF signals led to the understanding that respiratory sliding motion of the lung surface relatively to the LUS beam results in the formation of the DP peaks at the pleural line. When fluid-filled areas of the lung that result in formation of B-lines are moving across the LUS beam, the amplitude and phase of the wave reflected from the pleural line will change from one LUS pulse to the next, thus producing a peak in DP. Conversely, if the lung is fully aerated, its surface is uniformly reflective, and thus lung sliding relatively to LUS beam does not cause measurable changes in reflectance and associated DP peak.

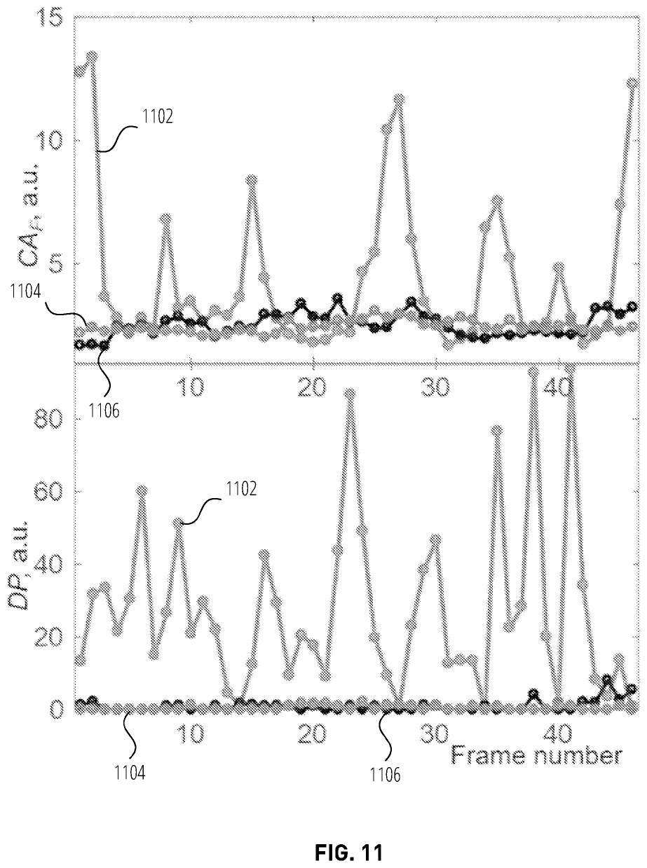

Signal amplitude variability at the longer time scale was characterized by changes in CA_{F} across the frames; representative examples of those changes for the datasets with no B-lines 1104, stationary B-lines 1106, and mobile B-lines 1102 are presented in FIG. 11. As seen, even in the case of relatively stationary B-lines the variability is larger than the case without B-lines, where signal amplitude consists of A-lines, reverberations, and other clutter. The changes of DP over the frames for the same three cases are shown in the lower chart in FIG. 11. It can be appreciated in the case of a mobile and transient B-line that the maximum in DP either precedes or follows the maximum of CA_{F}, but does not coincide with it, further supporting the observation that the peak in DP(d) at the pleural line forms due to the sliding of fluid-filled areas (and thus the B-lines) in and out of the LUS beam.

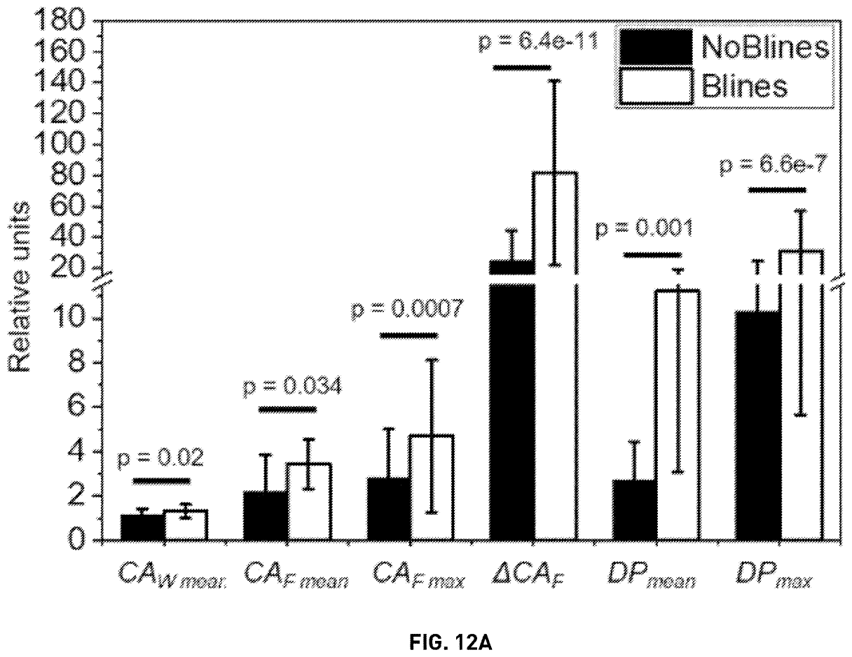

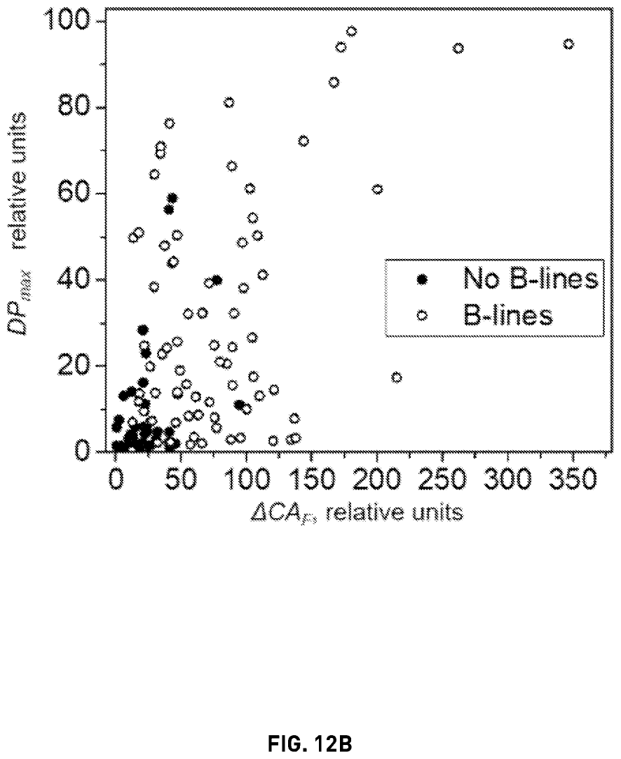

The results of the initial cumulative analysis of candidate B-line indicators are shown in FIG. 12A. All candidate indicators were statistically different between datasets with and without B-lines, although with different levels of significance and effect sizes. As seen, the indicators based on temporal variability of amplitude appeared superior in those respects, and thus ΔCA and DPₘₐₓ were selected for subsequent analysis. Individual data points corresponding to each LUS dataset are plotted in the ΔCA and DPₘₐₓ space in FIG. 12B. The points corresponding to the datasets without B-lines are clustered in the lower left corner of the plot. Thus, it was hypothesized that a combinatorial indicator of the form K₁ΔCA+K_DPₘₐₓ, rather than an individual indicator would have the highest predictive value for presence of B-lines.

Logistic regression was used to estimate the combined ability of ΔCA_{F} and DPₘₐₓ to predict the presence or absence of B-lines, as determined by expert consensus. Overall cross-validated AUC was 0.91 (95% CI=0.80-0.94) indicating excellent discrimination between true positives and true negatives. In order to calculate diagnostic test characteristics, a predictive probability threshold of 0.32 was selected by clinicians to optimize for high sensitivity to impaired pulmonary function, which is clinically useful in an emergency department and hospital setting. A subset of performance metrics for different predictive probability thresholds is shown in Table 1. At this threshold, the associated sensitivity was 93.2%, specificity was 58.5%, the positive predictive value (PPV) was 0.80, and the negative predictive value (NPV) was 0.83. The associated rates of true positives (TP), false positives (FP), false negatives (FN), and true negatives (TN) can be found in the contingency table (Table 2). Note that depending on the clinical context, a different threshold for classifying B-lines as positive or negative may be used.

TABLE 1
Performance of Signal Indicator-Based B-line
Classification Model at Different Thresholds

| Predictive probability | Sensitivity | Specificity |
|---|---|---|
| 0.1 | 100 | 7.3 |
| 0.2 | 98.6 | 41.5 |
| 0.3 | 94.5 | 58.5 |
| 0.7 | 69.9 | 87.8 |
| 0.8 | 64.4 | 87.8 |
| 0.9 | 47.3 | 90.2 |

TABLE 2
Classification Accuracy of Signal Indicator-
Based B-line Model (Threshold = 0.32)

Actual

| Predicted | No | Yes |
|---|---|---|
| No | TN: 24 (21.1%) | FN: 5 (4.4%) |
| Yes | FP: 17 (14.9%) | TP: 68 (59.7%) |
| Total (N) | 29 | 85 |

The complete disclosure of all patents, patent applications, and publications, and electronically available material cited herein are incorporated by reference in their entirety. Supplementary materials referenced in publications (such as supplementary tables, supplementary figures, supplementary materials, and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern.

The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, 10 when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

It will be appreciated that, although specific embodiments of the disclosure have been described herein for purposes of illustration, various modifications may be made without deviating from the spirit and scope of the disclosure. Accordingly, the disclosure is not limited except as by the claims.

### EXAMPLES

The following is a list of non-limiting example embodiments of the present disclosure.

Example 1. A computer-implemented method of monitoring health of a lung of a subject, the method comprising: receiving, by a computing device, at least one signal from an ultrasound sensor; processing, by the computing device, the at least one signal to detect an amount of a clinical sign of lung dysfunction; and presenting, by the computing device, a metric based on the detected amount of the clinical sign of lung dysfunction.

Example 2. The computer-implemented method of Example 1, wherein processing the at least one signal to detect the amount of the clinical sign of lung dysfunction includes: determining a pleural line reflectance variability.

Example 3. The computer-implemented method of Example 2, wherein determining the pleural line reflectance variability includes: determining a maximum Doppler power versus depth value for each sample of a plurality of samples; and determining a variability of the maximum Doppler power versus depth values.

Example 4. The computer-implemented method of Example 3, wherein determining the maximum Doppler power versus depth value for each sample of the plurality of samples includes normalizing each maximum Doppler power versus depth value using a Doppler threshold associated with the sample.

Example 5. The computer-implemented method of Example 4, wherein each sample is a set of Doppler pulses received after echo data.

Example 6. The computer-implemented method of any one of Examples 1-5, wherein processing the at least one signal to detect the amount of the clinical sign of lung dysfunction includes: determining a cumulative signal amplitude variability at a depth beneath a pleural line.

Example 7. The computer-implemented method of Example 6, wherein determining the cumulative signal amplitude variability includes, for each sample of a plurality of samples: determining an amplitude variability ΔA_{D} as:

$$Δ {A}_{D} = \frac{{A}_{min} - {A}_{max}}{{A}_{mean}}$$

wherein Aₘᵢₙ is a minimum amplitude, wherein Aₘₐₓ is a maximum amplitude, and wherein Aₘₑₐₙ is a mean amplitude.

Example 8. The computer-implemented method of Example 7, wherein the amplitude is determined as a sum of amplitude values between a start depth and an end depth.

Example 9. The computer-implemented method of Example 8, wherein the start depth is 9 centimeters from the ultrasound sensor, and wherein the end depth is 12 centimeters from the ultrasound sensor.

Example 10. The computer-implemented method of any one of Examples 7-9, wherein each sample is echo data.

Example 11. The computer-implemented method of Example 10, wherein the echo data is a B-mode image of a video.

Example 12. The computer-implemented method of any one of Examples 1-11, wherein processing the at least one signal to detect the amount of the clinical sign of lung dysfunction includes: determining a pleural line reflectance variability; determining a cumulative signal amplitude variability at a depth beneath a pleural line; and combining the pleural line reflectance variability and the cumulative signal amplitude variability to create a combinatorial indicator.

Example 13. The computer-implemented method of any one of Examples 1-12, wherein processing the signal to detect the amount of the clinical sign of lung dysfunction includes signal gating the signal from the ultrasound sensor using at least one other biosignal of lung function sensed from the subject; wherein the at least one other biosignal of lung function sensed from the subject includes at least one of a respiratory rate, an oxygen level, a pressure-volume trace, and an end-tidal carbon dioxide level.

Example 14. The computer-implemented method of any one of Examples 1-13, wherein processing the signal to detect the amount of the clinical sign of lung dysfunction includes: determining a signal characteristic for the received signal; and comparing the determined signal characteristic to at least one of a reference signal characteristic collected from a healthy lung, a reference signal characteristic collected from a lung exhibiting the clinical sign of lung dysfunction, and a reference signal characteristic collected from a simulated lung.

Example 15. The computer-implemented method of Example 14, further comprising: categorizing the lung health of the subject as healthy or unhealthy based on the detected amount of the clinical sign of lung dysfunction.

Example 16. The computer-implemented method of any one of Examples 14-15, wherein determining the signal characteristic for the received signal includes at least one of: measuring an amplitude of the received signal in a breath-to-breath timeframe; determining spectral components of the received signal in a breath-to-breath timeframe; performing doppler processing of the received signal in a breath-to-breath timeframe; and conducting a decorrelation analysis of the received signal in a breath-to-breath timeframe.

Example 17. The computer-implemented method of any one of Examples 1-16, wherein receiving the signal from the ultrasound sensor includes receiving signals from a plurality of ultrasound sensors, and wherein processing the signal to detect an amount of a clinical sign of lung dysfunction includes processing each signal from each ultrasound sensor of the plurality of ultrasound sensors to detect amounts of a clinical sign of lung dysfunction detected by each ultrasound sensor.

Example 18. The computer-implemented method of Example 17, wherein receiving the signals from the plurality of ultrasound sensors includes receiving the signals from the plurality of ultrasound sensors in parallel.

Example 19. The computer-implemented method of any one of Examples 17-18, wherein receiving the signals from the plurality of ultrasound sensors includes receiving the signals from the plurality of ultrasound sensors one at a time.

Example 20. The computer-implemented method of any one of Examples 17-19, wherein presenting the metric based on the detected amount of the clinical sign of lung dysfunction includes presenting separate metrics for each ultrasound sensor of the plurality of ultrasound sensors.

Example 21. The computer-implemented method of any one of Examples 17-20, wherein presenting the metric based on the detected amount of the clinical sign of lung dysfunction includes determining a combined metric for the plurality of ultrasound sensors and presenting the combined metric.

Example 22. The computer-implemented method of any one of Examples 1-21, wherein presenting the metric based on the detected amount of the clinical sign of lung dysfunction includes at least one of: transmitting, by the computing device, the metric to a display device for at least one of visual and auditory presentation; transmitting, by the computing device, the metric to a computer-readable storage medium for storage of longitudinal metric information for the subject; and transmitting, by the computing device, the metric to a medical treatment device for automatic adjustment of a setting of the medical treatment device based on the metric.

Example 23. A non-transitory computer-readable medium having computer-executable instructions stored thereon that, in response to execution by one or more processors of a computing device, cause the computing device to perform actions as recited in any one of Examples 1-22.

Example 24. A non-invasive system for monitoring lung health, the system comprising: a plurality of ultrasound sensors configured to be positioned on a thorax of a subject; and a monitoring computing system communicatively coupled to the plurality of ultrasound sensors, wherein the monitoring computing system includes logic that, in response to execution by the monitoring computing system, causes the monitoring computing system to perform actions comprising: receiving ultrasound signals from the plurality of ultrasound sensors; and processing the ultrasound signals to generate a metric usable for clinical diagnosis.

Example 25. The system of Example 24, wherein the metric usable for clinical diagnosis is a metric usable for determining at least one of a presence or absence of pneumothorax, a severity of pleural effusion, and a severity of pulmonary edema.

Example 26. The system of any one of Examples 24-25, wherein each ultrasound sensor of the plurality of ultrasound sensors includes: an anatomic cue to guide placement of the ultrasound sensor for monitoring a corresponding lung zone; and an adhesive layer configured to maintain acoustic coupling and contact between the ultrasound sensor and the subject.

Example 27. The system of any one of Examples 24-26, wherein each ultrasound sensor of the plurality of ultrasound sensors includes: a transceiver element; two quarter-wave matching layers; and circuitry for pulse generation and reception.

Example 28. The system of Example 27, wherein the transceiver element is a piezoelectric ceramic transducer.

Example 29. The system of any one of Examples 27-28, wherein each ultrasound sensor further includes a focusing lens.

Example 30. The system of any one of Examples 24-29, wherein the plurality of ultrasound sensors includes five ultrasound sensors for each lung to be monitored.

Example 31. The system of Example 30, wherein the five ultrasound sensors for each lung to be monitored are positioned on the thorax of the subject in an infraclavicular region, a mammary region, an axilla, an upper axillary region, and an infrascapular region.

Example 32. The system of any one of Examples 24-31, wherein the monitoring computing system is communicatively coupled to the plurality of ultrasound sensors via wired connections.

Example 33. The system of any one of Examples 24-32, wherein the monitoring computing system is communicatively coupled to the plurality of ultrasound sensors via wireless connections.

Example 34. The system of any one of Examples 24-33, wherein receiving the ultrasound signals from the plurality of ultrasound sensors includes concurrently receiving the ultrasound signals from the plurality of ultrasound sensors.

Example 35. The system of any one of Examples 24-34, wherein receiving the ultrasound signals from the plurality of ultrasound sensors includes receiving ultrasound signals from each ultrasound sensor of the plurality of ultrasound sensors in series.

Example 36. The system of any one of Examples 24-35, further comprising a display communicatively coupled to the monitoring computing system and configured to present an indication of the generated metric.

Example 37. The system of Example 36, wherein receiving ultrasound signals from the plurality of ultrasound sensors, processing the ultrasound signals to generate a metric usable for clinical diagnosis, and presenting an indication of the generated metric includes performing actions of a method as recited in any one of Example 1 to Example 22.

Example 38. The system of any one of Examples 24-37, further comprising a medical treatment device communicatively coupled to the monitoring computing system and configured to adjust an operational setting of the medical treatment device based on the generated metric.

Example 39. The system of Example 38, wherein the medical treatment device is a ventilator, an intravenous fluid dispenser, or a hemodialysis system.

Example 40. The system of any one of Examples 24-39, further comprising a non-transitory computer-readable medium communicatively coupled to the monitoring computing system, wherein the actions further comprise storing the generated metric on the non-transitory computer-readable medium to create a longitudinal record of the generated metric for the subject.

1. A computer-implemented method of monitoring health of a lung of a subject, the method comprising:
receiving, by a computing device, at least one signal from an ultrasound sensor;
processing, by the computing device, the at least one signal to detect an amount of a clinical sign of lung dysfunction; and
presenting, by the computing device, a metric based on the detected amount of the clinical sign of lung dysfunction.

2. The computer-implemented method of claim 1, wherein processing the at least one signal to detect the amount of the clinical sign of lung dysfunction includes:
determining a pleural line reflectance variability.

3. The computer-implemented method of claim 2, wherein determining the pleural line reflectance variability includes:
determining a maximum Doppler power versus depth value for each sample of a plurality of samples; and
determining a variability of the maximum Doppler power versus depth values.

4. The computer-implemented method of claim 3, wherein determining the maximum Doppler power versus depth value for each sample of the plurality of samples includes normalizing each maximum Doppler power versus depth value using a Doppler threshold associated with the sample.

5. The computer-implemented method of claim 4, wherein each sample is a set of Doppler pulses received after echo data.

6. The computer-implemented method of claim 1, wherein processing the at least one signal to detect the amount of the clinical sign of lung dysfunction includes:
determining a cumulative signal amplitude variability at a depth beneath a pleural line.

7. The computer-implemented method of claim 6, wherein determining the cumulative signal amplitude variability includes, for each sample of a plurality of samples:
determining an amplitude variability ΔA_{D} as:
$$Δ {A}_{D} = \frac{{A}_{min} - {A}_{max}}{{A}_{mean}}$$
wherein Aₘᵢₙ is a minimum amplitude,
wherein Aₘₐₓ is a maximum amplitude, and
wherein Aₘₑₐₙ is a mean amplitude.

8. The computer-implemented method of claim 7, wherein the amplitude is determined as a sum of amplitude values between a start depth and an end depth.

9-11. (canceled)

12. The computer-implemented method of claim 1, wherein processing the at least one signal to detect the amount of the clinical sign of lung dysfunction includes:
determining a pleural line reflectance variability;
determining a cumulative signal amplitude variability at a depth beneath a pleural line; and
combining the pleural line reflectance variability and the cumulative signal amplitude variability to create a combinatorial indicator.

13. The computer-implemented method of claim 1, wherein processing the signal to detect the amount of the clinical sign of lung dysfunction includes signal gating the signal from the ultrasound sensor using at least one other biosignal of lung function sensed from the subject;
wherein the at least one other biosignal of lung function sensed from the subject includes at least one of a respiratory rate, an oxygen level, a pressure-volume trace, and an end-tidal carbon dioxide level.

14. The computer-implemented method of claim 1, wherein processing the signal to detect the amount of the clinical sign of lung dysfunction includes:
determining a signal characteristic for the received signal; and
comparing the determined signal characteristic to at least one of a reference signal characteristic collected from a healthy lung, a reference signal characteristic collected from a lung exhibiting the clinical sign of lung dysfunction, and a reference signal characteristic collected from a simulated lung.

15. (canceled)

16. The computer-implemented method of claim 14, wherein determining the signal characteristic for the received signal includes at least one of:
measuring an amplitude of the received signal in a breath-to-breath timeframe;
determining spectral components of the received signal in a breath-to-breath timeframe;
performing doppler processing of the received signal in a breath-to-breath timeframe; and
conducting a decorrelation analysis of the received signal in a breath-to-breath timeframe.

17-21. (canceled)

22. The computer-implemented method of claim 1, wherein presenting the metric based on the detected amount of the clinical sign of lung dysfunction includes at least one of:
transmitting, by the computing device, the metric to a display device for at least one of visual and auditory presentation;
transmitting, by the computing device, the metric to a computer-readable storage medium for storage of longitudinal metric information for the subject; and
transmitting, by the computing device, the metric to a medical treatment device for automatic adjustment of a setting of the medical treatment device based on the metric.

23-24. (canceled)

25. A non-invasive system for monitoring lung health, the system comprising:
a plurality of ultrasound sensors configured to be positioned on a thorax of a subject; and
a monitoring computing system communicatively coupled to the plurality of ultrasound sensors, wherein the monitoring computing system includes logic that, in response to execution by the monitoring computing system, causes the monitoring computing system to perform actions comprising:
receiving ultrasound signals from the plurality of ultrasound sensors; and
processing the ultrasound signals to generate a metric usable for clinical diagnosis;
wherein processing the ultrasound signals to generate a metric usable for clinical diagnosis includes at least one of:
determining a pleural line reflectance variability; and
determining a cumulative signal amplitude variability at a depth beneath a pleural line.

26. The system of claim 25, wherein the metric usable for clinical diagnosis is a metric usable for determining at least one of a presence or absence of pneumothorax, a severity of pleural effusion, and a severity of pulmonary edema.

27. (canceled)

28. The system of claim 25, wherein each ultrasound sensor of the plurality of ultrasound sensors includes:
a transceiver element;
two quarter-wave matching layers; and
circuitry for pulse generation and reception.

29. (canceled)

30. The system of claim 28, wherein each ultrasound sensor further includes a focusing lens.

31-36. (canceled)

37. The system of claim 25, further comprising a display communicatively coupled to the monitoring computing system and configured to present an indication of the generated metric.

38. (canceled)

39. The system of claim 25, further comprising a medical treatment device communicatively coupled to the monitoring computing system and configured to adjust an operational setting of the medical treatment device based on the generated metric.

40. The system of claim 39, wherein the medical treatment device is a ventilator, an intravenous fluid dispenser, or a hemodialysis system.

41. (canceled)
